(19) United States
(12) Patent Application Publication
(10) Pub. No.: US 2023/0405901 A1
(43) Pub. Date: Dec. 21, 2023

(54) METHOD AND APPARATUS FOR MANUFACTURING A CUFFED MEDICAL DEVICE

(71) Applicant: TELEFLEX LIFE SCIENCES PTE. LTD., Singapore (SG)

(72) Inventor: Raamesh KRISHNAN, Perai (MY)

(21) Appl. No.: 18/461,177

(22) Filed: Sep. 5, 2023

Related U.S. Application Data
(63) Continuation of application No. PCT/IB2022/052096, filed on Mar. 9, 2022.
(60) Provisional application No. 63/160,387, filed on Mar. 12, 2021.

(51) Int. Cl. B29C 45/74; B29C 45/20; B29C 45/73; A61M 16/04

(52) U.S. Cl. CPC B29C 45/74; B29C 45/20; B29C 45/73; A61M 16/0443; B29L 2031/753

(57) ABSTRACT

A medical airway device having a cuff is manufactured by loading an injection molding apparatus with a thermosetting elastomeric material and shaping the cuff by injecting the material into an associated injection mold. The injection mold includes a sprue bush having a sprue passage in fluid communication with a mold cavity. The injection molding apparatus includes an injection nozzle that is positioned in contact with, or in close proximity to, the sprue bush. The thermosetting elastomeric material is discharged from the injection nozzle through the sprue passage and into the mold cavity. The cuff is formed by curing the thermosetting elastomeric material within the mold cavity and at the same time preventing premature curing of residual thermosetting elastomeric material within the injection nozzle by applying heat to the material within the mold cavity while cooling the injection nozzle with cooling fluid. The formed cuff is attachable to an airway tube.

### CROSS-REFERENCE TO RELATED APPLICATIONS

This application is a continuation of International Patent Application No. PCT/IB2022/052096, filed Mar. 9, 2022, which claims the benefit of U.S. Provisional Patent Application No. 63/160,387, filed Mar. 12, 2021, the content of which are hereby incorporated by reference in their entirety.

### FIELD OF THE INVENTION

The present invention generally relates to a method for manufacturing a cuffed medical device, such as a cuffed laryngeal mask airway, using a fluid-cooled injection molding nozzle.

### BACKGROUND

Cuffed medical devices are commonly used to form an airtight seal around a bodily passage or to block a bodily passage. Such cuffed medical devices include laryngeal mask airways and tracheal tubes, amongst others. For instance, a laryngeal mask airway (LMA) is a supraglottic device used to maintain the airway in patients, such as those undergoing general anesthesia. A conventional laryngeal mask airway includes an airway tube that connects to an elliptical mask with a cuff which is inserted through the patient's mouth and down the patient's windpipe. Once deployed, the cuff is inflated through a small auxiliary tube and forms an airtight seal on top of the glottis which allows a secure airway to be managed by a health care provider. A laryngeal mask airway is commonly used to channel oxygen or anesthesia gas to a patient's lungs during surgery, or during emergency care for an unconscious patient in a pre-hospital setting. Similarly, a tracheal tube is commonly used to maintain the airway of a patient. Conventional tracheal tubes include a flexible tube of rubber or plastic, and usually include an inflatable cuff around the distal end of the flexible tube. In use, the tracheal tube is introduced through the larynx into the trachea or windpipe, whereupon the cuff is inflated through a small auxiliary tube to seal against the wall of the patient's trachea.

For some conventional laryngeal mask airway devices, the mask portion is formed by adhering a foam material to both sides of a backplate. The foam forms an inflatable cuff that is attached to both sides of the plate. For some other conventional laryngeal mask airway devices, the mask portion is formed by attaching cuff members to the top and bottom of a backplate. The cuff members are often formed from flexible, resilient plastics material, such as polyvinyl chloride (PVC). A disadvantage of manufacturing these types of laryngeal mask airways is that the assembly of the mask portions requires a first step of fabricating the backplate and then a second step of adhering the cuff to the top and bottom of the plate, which can be both time consuming and costly.

Some medical device components may be manufactured by injection molding liquid silicone rubber (LSR). Manufacturing a laryngeal airway mask by such an injection molding process, for example, allows for simultaneously forming all parts of the mask portion. Moreover, medical devices formed from material such as liquid silicon rubber are often attractive due to their superior temperature range performance, chemical stability, and humidity resistance. For example, for a given volume of air, an inflatable cuff made from liquid silicone rubber will expand to a larger size than a comparable PVC cuff. This superior elasticity allows the liquid silicone rubber cuff to provide an anatomically superior seal with reduced mucosal pressure. Liquid silicone rubber cuffs are also able to deflate to a thickness that preserves flexural performance, but are also able to inflate such that that a thin cuff wall creates a satisfactory seal.

In conventional laryngeal mask airway devices, the inflatable cuff generally has an annular or elliptical toroid shape which can be difficult to manufacture. Some manufacturing processes employ injection molding techniques to form the inflatable cuff, as well as a backplate which is assembled in connection with an airway tube. It is often desired that the wall of the molded cuff be uniformly thin. In practice, however, injection of liquid rubber into the mold is under high pressure, which can cause thickness variations in the wall of the molded product. As a result, a relatively thick cuff wall is often formed so that small changes in wall thickness produced by movement of the core during the injection molding process will not result in deformities or rupture of the device when inflated during use. The difficulty with a relatively thick wall is that the inflatable cuff is less compliant in adapting itself to the contours of the human pharynx and larynx, thus reducing the efficacy of the desired seal to the laryngeal inlet, and/or unduly limiting the pressure that is possible to use for inflation of the lungs without loss of the airtight seal.

A further disadvantage of existing manufacturing processes is that they are highly labor-intensive, since the tubular airway portion of the device to which the peripheral cuff formation is attached must be connected via the backplate. This backplate is typically molded separately and forms an appropriate shape at one end to fit within the peripheral cuff, and at the other end to receive the tubular airway portion of the device. The backplate and the inner periphery of the inflatable cuff must be secured, such as by adhesive or the like, so as to complete the inflatable integrity of the cuff as well as the sealing effectiveness of the backplate to the inflatable cuff.

Another disadvantage of existing manufacturing processes is that conventional liquid silicone injection molding techniques yield a high reject rate for embedded defects on the surface of both the cuff and backplate portions of the laryngeal mask airway devices. Such embedded defects include rough or textured patches formed on the surface of the cuff and/or backplate, and are the result of heat transfer from the mold sprue bush to the injection molding nozzle, i.e., when the injection molding nozzle touches the mold sprue bush during an injection molding process. This is because the liquid silicone rubber injection molding process is considered a cold molding process, whereby there is no heat source connected to the injection molding device (from the mixer moving forward to the nozzle). Rather, only the mold is connected to a heat source for the purpose of curing the liquid silicone rubber into its desired shape. Thus, heat transfer occurs when the injection molding nozzle, which has a cold surface, contacts the mold sprue bush, which has a hot surface. Heat transfer may also occur when the injection molding nozzle is in proximity to the hot sprue bush. Because the injection nozzle is meant to be colder than the mold sprue bushing, a problem that occurs is that heat transfer from the bushing to the nozzle causes the injected material to cure too early while still in the nozzle tip, or as residual material is left in the nozzle tip. As illustrated in FIG. 1, this heat transfer problem from the sprue bush 10 impacts the viscosity of the material inside the nozzle 20 and contributes to the embedded defects.

Moreover, the transfer of heat from such a hot medium to a cold medium happens each cycle of the injection molding process. As a result, the cumulative effect of each contact between the cold injection molding nozzle and the hot mold sprue bush will gradually increase the temperature of the injection molding nozzle to certain degree. The liquid silicone rubber material that is leftover on the tip of the nozzle will prematurely cure and subsequently be ejected out as embedded material formed together with finished product. For example, the prematurely cured material will become embedded on the surface of the molded cuff or backplate.

Other problems caused by this transfer of heat to the injection nozzle include the need for frequent cleaning of hardened injection molding material from the injection pipeline, including the injection nozzle as well as the material mixer, in order to remove blockages within the injection pipeline. Moreover, once an embedded defect occurs on the cuff or backplate, the injection molding process must be stopped so that the operator can troubleshoot the problem, which may result in the operator cleaning the injection nozzle or purging material from the injection pipeline which can be both time consuming and costly, as it interrupts regularly scheduled cleaning plans.

Accordingly, there is a clear and substantial need for a new and improved method of manufacturing a cuffed medical device, such as a laryngeal mask airway, via injection molding while eliminating or decreasing instances of embedded defects caused from unwanted heat transfer between the mold sprue bushing and the injection nozzle, among other advantages. The method of manufacturing a cuffed medical device according to the present invention solves the above mentioned problems by preventing or mitigating the occurrence of heat transfer from the mold sprue bush to the injection nozzle by using a water cooling system to circulate the heat each time the bushing contacts, or is in proximity to, the nozzle, thus maintaining the cold temperature of the nozzle surface. In particular, the method of manufacturing according to the present invention utilizes cooling channels formed in the nozzle which keep it at sufficiently low temperature to prevent curing of injection molding material at its tip.

### SUMMARY

The foregoing needs are met, to a great extent, by the present invention of a method of manufacturing a medical airway device having a cuff, the method comprising providing an injection molding apparatus loaded with a thermosetting elastomeric material, the injection molding apparatus comprising an injection nozzle; providing an injection mold comprising a sprue bush and a mold cavity, the sprue bush defining a sprue passage in fluid communication with the mold cavity; positioning the injection nozzle relative to the sprue bush such that a portion of the injection nozzle contacts, or is in proximity to, a portion of the sprue bush; shaping the cuff by injecting the thermosetting elastomeric material from the injection nozzle through the sprue passage and into the mold cavity; forming the cuff by curing the thermosetting elastomeric material within the mold cavity and at the same time preventing premature curing of residual thermosetting elastomeric material within the injection nozzle by applying heat to the thermosetting elastomeric material within the mold cavity while cooling the injection nozzle with a cooling fluid; and attaching the formed cuff to an airway tube.

According to another aspect of the present invention, the injection nozzle comprises a proximal end, a distal end, and a nozzle passage longitudinally extending from the proximal end to the distal end, nozzle passage terminating at a nozzle outlet at the distal end.

According to another aspect of the present invention, the injection nozzle comprises a cooling channel in which the cooling fluid flows for removing heat from the injection nozzle and preventing curing of the thermosetting elastomeric material within the injection nozzle.

According to another aspect of the present invention, the cooling channel includes a fluid inlet and a fluid outlet, the fluid inlet being in fluid communication with a fluid supply source.

According to another aspect of the present invention, the fluid inlet and the fluid outlet are disposed in the injection nozzle adjacent the proximal end.

According to another aspect of the present invention, a portion of the cooling channel flows along a portion of the nozzle passage.

According to another aspect of the present invention, a portion of the cooling channel surrounds a portion of the nozzle passage.

According to another aspect of the present invention, the nozzle passage is tapered in a direction from the proximal end of the injection nozzle toward the nozzle outlet at the distal end of the injection nozzle.

According to another aspect of the present invention, the proximal end of the injection nozzle is configured to threadedly engage a manifold of the injection molding apparatus.

According to another aspect of the present invention, the injection mold further comprises a mold core defining a peripheral space within the mold cavity, the peripheral space corresponding to a shape of the cuff.

According to another aspect of the present invention, the formed cuff is inflatable.

According to another aspect of the present invention, the injection molding apparatus further comprises a manifold containing the thermosetting elastomeric material.

According to another aspect of the present invention, a heat source is connected to the injection mold.

According to another aspect of the present invention, the medical airway device is a tracheal tube.

According to another aspect of the present invention, the medical airway device is a laryngeal mask airway.

According to another aspect of the present invention the method further comprises shaping a backplate by injecting the thermosetting elastomeric material from the injection nozzle through the sprue passage and into the mold cavity; forming the backplate by curing the thermosetting elastomeric material within the mold cavity and at the same time preventing premature curing of residual thermosetting elastomeric material within the injection nozzle by applying heat to the thermosetting elastomeric material within the mold cavity while cooling the injection nozzle with a cooling fluid; and attaching the formed backplate to the airway tube.

According to another aspect of the present invention, the injection mold further comprises a mold core defining a space within the mold cavity corresponding to a shape of the backplate.

According to another aspect of the present invention, injection molding the cuff and the backplate are formed in a single step.

According to another aspect of the present invention, the thermosetting elastomeric material comprises liquid silicone rubber.

According to another aspect of the present invention, the cooling fluid comprises water.

There has thus been outlined certain embodiments of the present invention in order that the detailed description thereof herein may be better understood, and in order that the present contribution to the art may be better appreciated. There are additional embodiments of the present invention that will be described below and which form the subject matter of the claims appended hereto.

In this respect, before explaining at least one aspect of the method of manufacturing a cuffed medical device in detail, it is to be understood that the method of manufacturing is not limited in its application to the details of construction and to the arrangements of the components set forth in the following description or illustrated in the drawings. The method of manufacturing a cuffed medical device is capable of aspects in addition to those described, and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this invention is based may readily be utilized as a basis for the designing of other structures, methods, and systems for carrying out the several purposes of the method of manufacturing a cuffed medical device. It is important, therefore, that the claims be regarded as including such equivalent constructions insofar as they do not depart from the spirit and scope of the invention.

### BRIEF DESCRIPTION OF THE DRAWINGS

In order that the present invention may be readily understood, aspects of the method of manufacturing a cuffed medical device are illustrated by way of examples in the accompanying drawings, in which like parts are referred to with like reference numerals throughout.

FIG. 1 illustrates a conventional nozzle and sprue bush.

FIG. 2 illustrates an implementation of a cuffed medical airway device made by the manufacturing process of the present invention.

FIG. 3 illustrates a perspective view of a mask portion of the medical airway device of FIG. 2.

FIG. 4 illustrates a cross-sectional side view of the mask portion of the medical airway device of FIG. 2.

FIG. 5 illustrates a cross-sectional side view of an implementation of a mold used in the manufacturing process of the present invention.

FIG. 6 illustrates a cross-sectional side view of an implementation of a mask portion of the medical airway device formed using the mold of FIG. 5.

FIG. 7 illustrates a injection nozzle used in the manufacturing process of the present invention.

FIG. 8 illustrates a cross-sectional side view of another implementation of a mold used in the manufacturing process of the present invention.

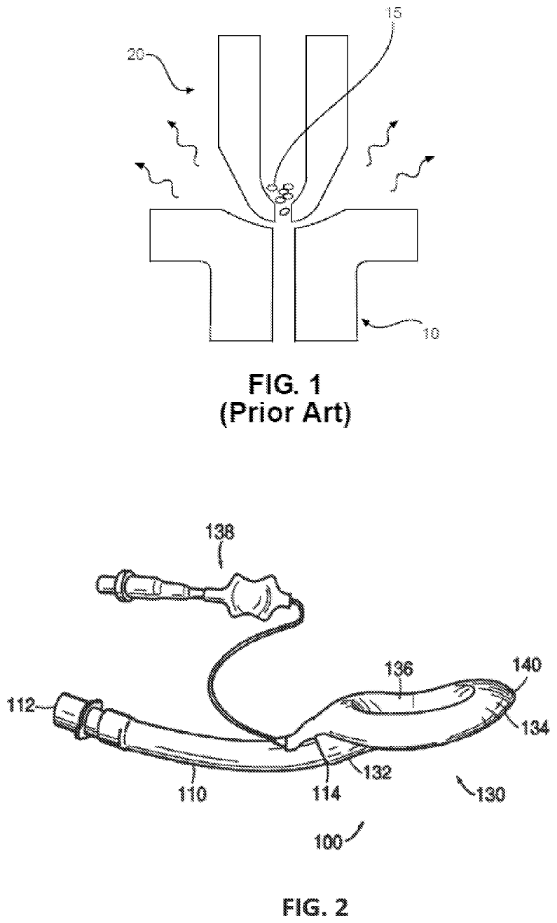

### DETAILED DESCRIPTION

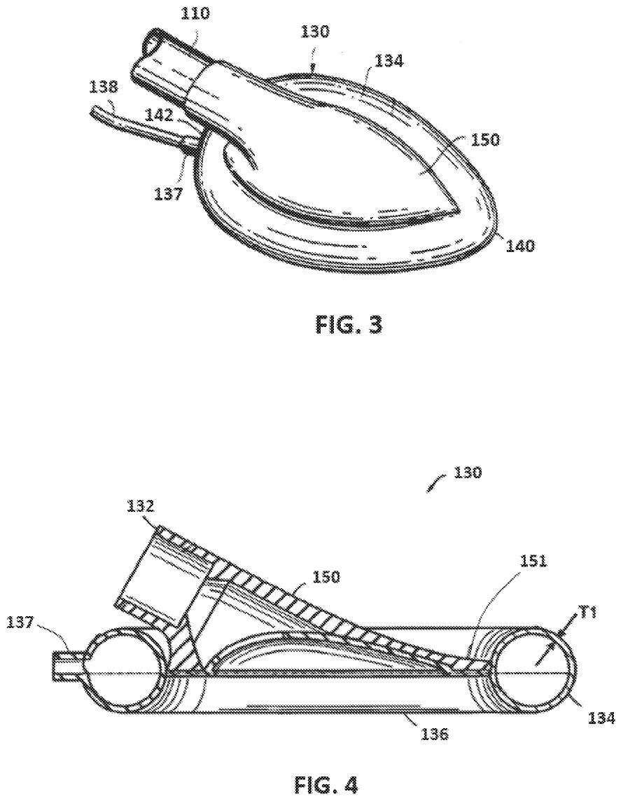

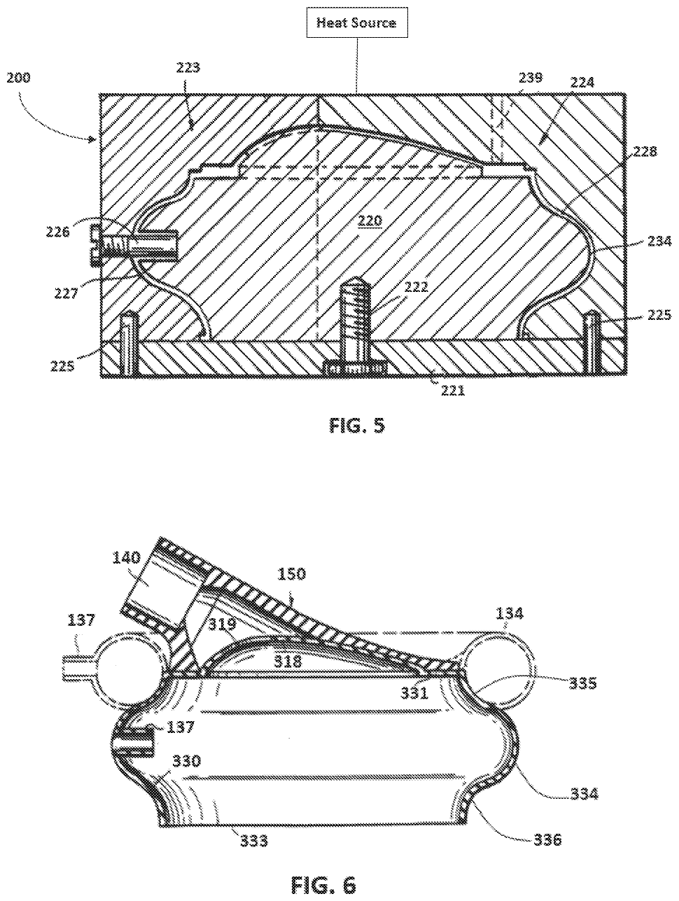

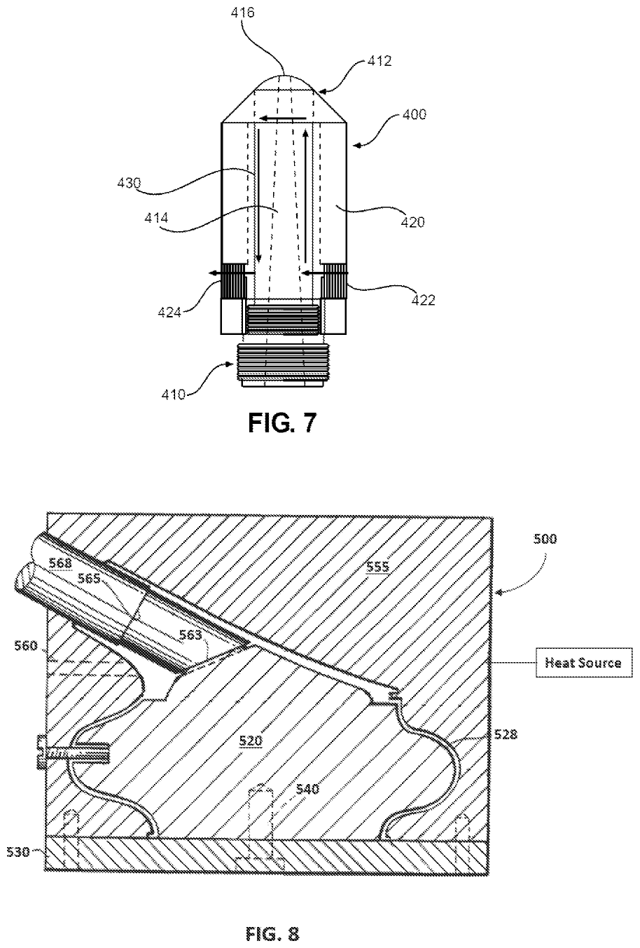

The present invention describes a method of manufacturing a cuffed medical device, such as a laryngeal mask airway or a tracheal tube. An example of such a cuffed laryngeal mask airway device 100 manufactured in accordance with the present invention is depicted in FIGS. 2 and 3. The laryngeal mask airway 100 includes a flexible cylindrical breathing or airway tube 110 and a mask portion 130. The breathing tube 110 extends from a proximal end 112 to a distal end 114, and the mask portion 130 is coupled to the tube's distal end 114. The mask portion 130 includes a breathing tube connector 132 connected to a backplate 150, which is connected to an inflatable cuff 134 having a generally elliptical toroid shape. In other embodiments, the inflatable cuff may have a generally annular toroid shape, amongst others. The mask portion 130 also defines a central passageway extending from the breathing tube connector 132 to an opening 136 surrounded by the cuff 134. The distal end 114 of the breathing tube 110 is telescopically connected to the breathing tube connector 132 of the mask portion 130. The laryngeal mask airway device 100 provides a continuous sealed airway extending from the proximal end 112 of the breathing tube 110 to the opening 136. A supply tube 138 is provided for selectively inflating or deflating the cuff 134.

During use, the cuff 134 is deflated so that the mask portion 130 can be inserted through the patient's mouth and into the patient's pharynx, with the proximal end 112 of the breathing tube 110 accessible for ventilation via or outside the patient's mouth. The mask portion 130 is preferably positioned so that a distal end 140 of cuff 134 rests against the patient's normally closed esophagus and so that the opening 136 defined by the cuff 134 is aligned with the entryway of the patient's trachea (i.e., the patient's glottic opening). After the mask portion is so positioned, the cuff 134 is inflated, thereby forming a seal around the patient's glottic opening which establishes a sealed airway extending from the proximal end 112 of the breathing tube 110 to the patient's trachea.

FIG. 4 shows a cross-sectional side view of the mask portion 130 of the laryngeal mask airway device 100. As illustrated, the mask portion 130 includes the inflatable cuff 134 and the backplate 150. The backplate 150 includes the breathing tube connector 132 for receiving, or coupling to, the cylindrical airway tube 110. The backplate defines a sealed passageway, or airway, that extends from the breathing tube connector 132 to the opening 136 peripherally surrounded by the cuff 134. The thickness T1 of the cuff wall is in the range 0.4 to 1.0 millimeters, and preferably about 0.7-0.8 millimeters.

The mask portion 130 comprises a first molded part, such as the inflatable cuff 134, and a second molded part, such as the backplate 150. The generally elliptical and toroid-shaped inflatable cuff 134 surrounds the periphery of the backplate 150. A distal end 140 of the cuff 134 may be pointed, and the proximal end 142 of the cuff may be rounded, to facilitate ease of insertion into the patient's airway when the elliptical cuff 134 is deflated, as well as to ensure proper placement of the laryngeal mask portion 130 over the esophageal inlet when the cuff is inflated. Such inflation and deflation of the cuff 134 may be performed via the supply tube 138, which is connected to a supply tube connector 137 at the proximal end 142 of the cuff 134. The supply tube 138 is operable to provide a supply of air or other fluid, including gas or liquid, to the cuff 134 for inflation. The pointed distal end 140 of the cuff 134 and the rounded proximal end 142 of the cuff may generally conform with similar features of a base rim 151 of the backplate 150. In some implementations, a flexible membrane may be provided to close the lumen of the mask, except for a passage established by one or more openings for preventing the epiglottis from blocking the airway passage of the mask.

In accordance with the present invention, a cuffed medical device, such as the laryngeal mask airway 100, is manufactured by injection molding thermosetting elastomeric materials, such as liquid silicone rubber, into desired shapes. In particular, the manufacturing method of the present invention employs injection molding liquid silicone rubber to form the inflatable cuff 134, either alone or together with the backplate 150, which is then assembled in connection with the airway tube 110. An advantage of using such elastomeric materials is that they are strong and durable, yet sufficiently flexible for insertion into a patient's airway. More particularly, forming the inflatable cuff and/or the backplate of the laryngeal mask airway by injection molding liquid silicone rubber is advantageous due to its wide ranging temperature performance, chemical stability, and humidity resistance. The elasticity of the liquid silicone rubber cuff provides an anatomically airtight seal with reduced mucosal pressure when the cuff is inflated. Such a liquid silicone rubber cuff is also able to deflate to a thickness that preserves flexural performance during insertion into a patient's airway.

FIG. 5 illustrates an example of a mold 200 operable for use in the injection molding process of the present invention. In particular, when manufacturing the mask portion 130 of the laryngeal mask airway device 100, the mold 200 is used to fabricate and shape the inflatable cuff 134. The mold 200 includes a cavity defined by a mold shell configured to cooperate with a core 220 which is secured to a mold base or plate 221 by one or more bolts 222. In some aspects, the core 220 may be securely clamped to the mold base 221. The mold shell may be similarly secured to the mold base 221. For example, the mold shell may comprise first and second separable mold shell portions 223, 224 configured to be secured to the mold base 221 by respective dowel pins 225. In other aspects, the first and second mold shells 223, 224 may be clamped to each other. A threaded removable pin 226 may be provided in one of the mold shells 223, such that the pin 226 enters a local bore 227 in the core 220 in order to integrally form the supply tube connector 137 at the proximal end 142 of the cuff 134 during the injection molding process.

A peripheral space 228 between the core 220 and the cooperating mold shells 223, 224 accounts for an intermediate molded product from which the cuff 134 of the mask portion 130 of the laryngeal mask airway device 100 is subsequently formed. For instance, the liquid silicone rubber is discharged from an injection molding nozzle and into the peripheral space 228 of the mold 200 via one or more sprue passages 239, which may be defined by a sprue bush in or attached to the mold, thereby forming the intermediate molded cuff. While the sprue passage 239 is shown vertically disposed in the corresponding mold shell, it should be appreciated that in some implementations the sprue passage may be horizontally disposed in the mold shell. The intermediate molded cuff product is then cured sufficiently for removal from the mold. Such curing is accomplished by applying heat to the mold from a heat source. The intermediate molded cuff product is then stripped from the mold 200 by peeling it from the core 220.

As shown in FIG. 6, the intermediate molded cuff product comprises a thin-walled skirt 330 depending from an essentially flat but thicker elliptical or annular flange 331 to the upper inner edge of which a thin-walled shallow dome 318 defining the flexible membrane effectively closes the lumen within the flange 331, with the exception of plural apertures 319 provide near the proximal end of the dome. The skirt 330 is flexible and stretchable, and comprises a peripherally continuous lower rim 333 of essentially the same peripheral extent as the outer edge of the flange 331. Between the flange 331 and the lower rim 333, the molded skirt 330 features a peripherally continuous radially outward convex bulge 334 which becomes concave near its juncture with the annular flange 331 and near the lower rim 333. This convex bulge 334 in the skirt 330 is formed by a corresponding bulge 234 section of the peripheral space 228 in the mold 200. At the upper concave portion 335, a juncture with the flange 331 is substantially perpendicular to the plane of the flange 331. At the lower concave portion 336, a termination at the lower rim 333 is substantially perpendicular to the lower plane defined by rim 333.

Inverting or reversing the flexible skirt 330 converts the convex bulge 334 into an outer concave contour which is smoothly contiguous to the upper and lower concave portions 335, 336, thereby forming the inflatable cuff 134 of the mask portion 130. Also during this process of inverting the skirt 330, the inwardly directed tube connector 137 of the molded skirt 330 is also reversed to project outwardly, thereby becoming operable to connect to the supply tube 138 for inflating or deflating the cuff 134. Moreover, the molded longitudinal length spanning the lower end of the skirt 330 is equal to or less than the corresponding longitudinal length spanning the upper surface of the flange 331. This relationship assures a natural fit of the flange 331 to the lower rim 333 upon inversion of the skirt 330. In some aspects, the engageable surfaces of the flange 331 and the lower rim 333 may be coated with suitable silicone adhesive to ensure a secure connection therebetween upon reversal of the skirt 330.

The mask portion 130 may then be completed by assembling a separately molded backplate 150 to the cuff 134. The backplate 150 may be molded from liquid silicone rubber material as similarly used in injection molding the cuff 134. A bottom surface of the backplate 150 has a generally flat elliptical or annular shape conforming to the profile of the flat upper surface of the flange 331, and may be precoated with a suitable adhesive for securing to the flange 331 once they are assembled together. The breathing tube connector 132 of the backplate 150 includes a cylindrical counterbore 140 configured to connect to the airway tube 110. The supply tube connector 137 of the inflatable cuff 134 is configured to connect to the supply tube 138. In some aspects, a bead of silicone adhesive may be applied to a peripherally continuous groove located between an outer exposed contoured portion of the backplate 150 and cuff 134 (i.e., the now-inverted but previously lower rim 333 of the skirt 330) to provide additional securement therebetween when the cuff is deflated for ease of insertion of the mask portion 130 into, and removal from, a patient's airway.

FIG. 7 depicts an exemplary injection molding nozzle 400 used in the manufacturing method of the present invention. The nozzle 400 is configured for use with a sprue bush in or attached to the mold. The nozzle includes a proximal end 410, a distal end 412, and a main channel 414 extending longitudinally from the proximal end to the distal end. The proximal end 410 of the nozzle 400 is in fluid communication with an injection molding apparatus. Such an injection molding apparatus may comprise a material mixer, a material manifold, a flow meter, and a mold heater, amongst other components. The proximal end 410 of the nozzle may be configured for a threaded connection to the material manifold.

The distal end 412 of the nozzle 400 includes an outlet 416 configured to discharge uncured liquid silicone rubber from the nozzle to the associated mold cavity via the sprue bush. The main injection channel 414 has a tapered diameter extending from the proximal end 410 of the nozzle toward the distal end 412, such that a reduced diameter portion ends at the nozzle outlet 416. The tapered discharge end of the nozzle is configured to abut the mold sprue bush to permit ejection of the injection molding material into the sprue passage of the mold. In some implementations, the liquid silicone rubber residing in the material manifold is forced into the nozzle under the pressure generated by an injection barrel screw or a piston of the injection molding apparatus. The nozzle may be sealed to the manifold by one or more elastomeric gaskets, such as rubber washers or O-rings, to prevent fluid leakage.

The mold is connected to the mold heater for heat curing the liquid silicone rubber into its desired shape during the injection molding process. The nozzle 400 is configured to be cooled to ensure that the uncured injection molding material within the nozzle remains in the uncured state during the heating or curing phase of the molding cycle (i.e., when the molding material is received within the mold cavity and heat cured to provide the solidification necessary to produce the molded cuff and/or backplate). Such cooling of the nozzle is desired due to the thermal conductivity of the material of the nozzle and the sprue bush, which may both be made from steel or other metal. For example, the metal-to-metal contact between the nozzle and the sprue bush of the injection mold facilitates heat transfer between the sprue bush and the nozzle during the injection molding process. Close proximity between the nozzle and the sprue bush of the injection mold also facilitates heat transfer between the sprue bush and the nozzle during the injection molding process. This heat transfer problem impacts the viscosity of the injection molding material inside the nozzle. In particular, such a transfer of heat from a hot sprue bush to the nozzle tip 416 results in premature curing of the liquid silicone rubber material within the nozzle. For example, prematurely cured liquid silicone rubber leftover in the nozzle tip is caused from the transfer of heat from the mold sprue bush to the nozzle, which can lead to embedded defects, such as rough or textured patches, formed on the surface of the molded cuff and/or backplate.

During the injection molding process, the mold is heated by the mold heater to a temperature between 160° C. to 220° C. for heat curing the associated liquid silicone rubber within the mold cavity. As the liquid silicone rubber in the mold is cured, the leftover liquid silicone rubber disposed within the nozzle waiting for the next injection and cure cycle is adversely heated by thermal energy conducted from the mold sprue bush to the portion of the nozzle, such as the nozzle tip that is in contact with, or in proximity to, the mold sprue bush. To alleviate this problem, the nozzle is configured to cool the uncured liquid silicone rubber residing within the nozzle 400 to prevent premature curing of the material due to heat transfer from the mold sprue bush to the nozzle when the sprue bush and the nozzle contact each other, or are in proximity to each other, during the injection molding process.

Accordingly, the present method of manufacturing the cuff and/or backplate comprises maintaining a pre-cure temperature level for the uncured injection molding material resident within the nozzle. The nozzle maintains such a pre-cure temperature by flowing a cooling fluid, such as cold water, through one or more cooling channels 420 machined or cast into the nozzle body so that the cooling channel surrounds or jackets the main nozzle channel 414. In one implementation, the one or more cooling channels 420 provided within the nozzle are filled with cold water that flows through the nozzle alongside the main passage 414 from a fluid inlet 422 near the proximal end of the nozzle, toward the distal end 412 of the nozzle, and then back toward the proximal end from where the fluid exits via a fluid outlet 424. The flow of cooling fluid 430, such as water, through the body of the nozzle is generally parallel to the direction of flow of the liquid silicone rubber material within the main channel 414 and serves to cool the nozzle and the associated liquid silicone rubber within the main passage 414 from the influx of heat passed to the nozzle from the mold sprue bush. In some aspects, the flow of cooling fluid also surrounds a portion of the nozzle passage 414.

Accordingly, the water cooling channel 420 maintains the primary injection channel 414 at a low temperature over its entire length, including at the outlet 416. The cold water is introduced into the cooling channel 420 via a fluid supply connected to the fluid inlet 422. Heat transferred to the nozzle from the mold sprue bush is thus further transferred to the cold water in the fluid channel 420 as it flows through the body of the nozzle. The fluid cooling channel 420 maintains the temperature of the injection molding material within the nozzle at a temperature generally between 5° C. and 50° C. so as to avoid early curing of the injection molding material within the nozzle prior to it being injected into the mold sprue bush.

FIG. 8 illustrates another example of a mold 500 operable for use in the injection molding process of the present invention. This mold 500 enables one-step molding of the mask portion 130, in which the backplate 150 is integrally molded to the cuff 134 during the injection molding process. The mold 500 includes a single cavity defined by a mold shell 555. The mold 500 comprises a core 520 disposed within the cavity and retained to a mold base or plate 530 by one or more bolts 540. A peripheral space 528 between the core 520 and the mold shell 555 accounts for an intermediate molded product from which the cuff of the mask portion of the laryngeal mask airway device is subsequently formed.

The peripheral space 528 also accounts for the molded backplate. In particular, the core 520 rises with a convergent upper body portion 563 which is profiled to establish internal features of the back-plate. The upper body portion 563 is configured to fit with a mold-cavity defining plug 568 which is retractably guided in a bore of the outer-mold shell 555. The plug 568 may include a shoulder 565 that defines a limit stop formed in the counterbore portion of the backplate for snugly receiving the airway tube when the laryngeal mask airway device is fully assembled. During the injection molding process, liquid molding material, such as liquid silicone rubber, is discharged from an injection molding nozzle and into the peripheral space 528 of the mold 500 via one or more sprue passages 560 in the mold, thereby forming both the molded backplate and the intermediate molded cuff. The sprue passage may be defined by a sprue bushing formed in, or attached to, the mold. While the sprue passage 560 is shown horizontally disposed in the corresponding mold shell, it should be appreciated that in some implementations the sprue passage may be vertically disposed in the mold shell. The inflatable cuff is then formed by folding up the intermediate molded cuff product in the same manner as previously described in detail above. Curing of the molded cuff and backplate is accomplished by applying heat to the mold from a heat source.

While a method of manufacturing a cuffed medical device has been described in terms of what may be considered to be specific aspects, the present invention is not limited to the disclosed aspects. Additional modifications and improvements to the method of manufacturing the cuffed medical device may be apparent to those skilled in the art. Moreover, the many features and advantages of the disclosure are apparent from the detailed specification, and thus, it is intended by the appended claims to cover all such features and advantages of the present invention which fall within the spirit and scope of the disclosure. Further, it is not desired to limit the disclosure to the exact construction and operation illustrated and described, and accordingly, all suitable modifications and equivalents may be resorted to, falling within the scope of the disclosure. The present disclosure should therefore be considered as illustrative and not restrictive. As such, this disclosure is intended to cover various modifications and similar arrangements included within the spirit and scope of the claims, which should be accorded their broadest interpretation so as to encompass all such modifications and similar structures.

What is claimed is:

1. A method of manufacturing a medical airway device having a cuff, the method comprising:
providing an injection molding apparatus loaded with a thermosetting elastomeric material, the injection molding apparatus comprising an injection nozzle;
providing an injection mold comprising a sprue bush and a mold cavity, the sprue bush defining a sprue passage in fluid communication with the mold cavity;
positioning the injection nozzle relative to the sprue bush such that a portion of the injection nozzle contacts, or is in proximity to, a portion of the sprue bush;
shaping the cuff by injecting the thermosetting elastomeric material from the injection nozzle through the sprue passage and into the mold cavity;
forming the cuff by curing the thermosetting elastomeric material within the mold cavity and at the same time preventing premature curing of residual thermosetting elastomeric material within the injection nozzle by applying heat to the thermosetting elastomeric material within the mold cavity while cooling the injection nozzle with a cooling fluid; and
attaching the formed cuff to an airway tube.

2. The method of manufacturing according to claim 1, wherein the injection nozzle comprises a proximal end, a distal end, and a nozzle passage longitudinally extending from the proximal end to the distal end, the nozzle passage terminating at a nozzle outlet at the distal end.

3. The method of manufacturing according to claim 2, wherein the injection nozzle comprises a cooling channel in which the cooling fluid flows for removing heat from the injection nozzle and preventing curing of the thermosetting elastomeric material within the injection nozzle.

4. The method of manufacturing according to claim 3, wherein the cooling channel includes a fluid inlet and a fluid outlet, the fluid inlet being in fluid communication with a fluid supply source.

5. The method of manufacturing according to claim 4, wherein the fluid inlet and the fluid outlet are disposed in the injection nozzle adjacent the proximal end.

6. The method of manufacturing according to 3, wherein a portion of the cooling channel flows along a portion of the nozzle passage.

7. The method of manufacturing according to claim 3, wherein a portion of the cooling channel surrounds a portion of the nozzle passage.

8. The method of manufacturing according to claim 2, wherein the nozzle passage is tapered in a direction from the proximal end of the injection nozzle toward the nozzle outlet at the distal end of the injection nozzle.

9. The method of manufacturing according to claim 2, wherein the proximal end of the injection nozzle is configured to threadedly engage a manifold of the injection molding apparatus.

10. The method of manufacturing according to claim 1, wherein the injection mold further comprises a mold core defining a peripheral space within the mold cavity, the peripheral space corresponding to a shape of the cuff.

11. The method of manufacturing according to claim 1, wherein the formed cuff is inflatable.

12. The method of manufacturing according to claim 1, wherein the injection molding apparatus further comprises a manifold containing the thermosetting elastomeric material.

13. The method of manufacturing according to claim 1, further comprising a heat source connected to the injection mold.

14. The method of manufacturing according to claim 1, wherein the medical airway device is a tracheal tube.

15. The method of manufacturing according to claim 1, wherein the medical airway device is a laryngeal mask airway.

16. The method of manufacturing according to claim 1, further comprising
shaping a backplate by injecting the thermosetting elastomeric material from the injection nozzle through the sprue passage and into the mold cavity;
forming the backplate by curing the thermosetting elastomeric material within the mold cavity and at the same time preventing premature curing of residual thermosetting elastomeric material within the injection nozzle by applying heat to the thermosetting elastomeric material within the mold cavity while cooling the injection nozzle with a cooling fluid; and
attaching the formed backplate to the airway tube.

17. The method of manufacturing according to claim 16, wherein the injection mold further comprises a mold core defining a space within the mold cavity corresponding to a shape of the backplate.

18. The method of manufacturing according to claim 16, wherein injection molding the cuff and the backplate are formed in a single step.

19. The method of manufacturing according to claim 1, wherein the thermosetting elastomeric material comprises liquid silicone rubber.

20. The method of manufacturing according to claim 1, wherein the cooling fluid comprises water.
